(19) United States
(12) Patent Application Publication
(10) Pub. No.: US 2020/0206174 A1
(43) Pub. Date: Jul. 2, 2020

(54) TREATMENT AND PREVENTION OF NEURONAL CELL LOSS USING L-ORNITHINE IN COMBINATION WITH AT LEAST ONE OF PHENYLACETATE AND PHENYLBUTYRATE

(71) Applicant: OCERA THERAPEUTICS, INC., Redwood City, CA (US)

(72) Inventors: Christopher F. Rose, Montreal, Quebec (CA); Marc-André Clément, Montreal, Quebec (CA); Cristina R. Bosoi, Montreal, Quebec (CA); Mariana Macedo Oliveira, Montreal, Quebec (CA); Mélanie Tremblay, Montreal, Quebec (CA); Chantal Bémeur, Montreal, Quebec (CA)

(21) Appl. No.: 15/751,442

(22) PCT Filed: Sep. 22, 2016
(86) PCT No.: PCT/US2016/053176

§ 371 Date: Feb. 8, 2018

Related U.S. Application Data
(60) Provisional application No. 62/233,002, filed on Sep. 25, 2015.

(51) Int. Cl. A61K 31/216; A61K 31/192; A61K 31/198; A61P 25/00; A61P 1/16; A61P 9/02

(52) U.S. Cl. CPC A61K 31/216; A61K 31/192; A61P 9/02; A61P 25/00; A61P 1/16; A61K 31/198

(57) ABSTRACT

Disclosed herein are methods of treating and preventing neuronal cell loss in a subject using ornithine in combination with phenylacetate or phenylbutyrate. In some embodiments, the subject has received or will receive a surgical procedure (for example, liver transplantation) for treating a liver disease. In some embodiments, the subject suffers from a liver disease and hypotension.

### RELATED APPLICATIONS

The present application claims priority under 35 U.S.C. § 119(e) to U.S. Provisional Application No. 62/233,002, filed on Sep. 25, 2015. The content of this related application is herein expressly incorporated by reference in its entirety.

### BACKGROUND

### Field

The present application relates to the fields of pharmaceutical chemistry, biochemistry and medicine. One aspect relates to the treatment and prevention of neuronal cell loss using ornithine in combination with at least one of phenylacetate and phenylbutyrate.

### Description of the Related Art

Neurons, also known as neuronal cells, are highly specialized cells of the nervous system. Neurons are electrically excitable cells that process and transmit information through electrical and chemical signals. Liver diseases are often accompanied with neuropsychiatric complications characterized by cognitive and motor dysfunction. The only curative treatment for end-stage liver diseases and hepatic encephalopathy (HE) to date remains to be liver transplantation (LT).

### SUMMARY

Disclosed herein is a method of treating a condition of neuron loss. The method, in some embodiments, comprises administering ornithine in combination with at least one of phenylacetate and phenylbutyrate to a subject in need thereof, and thereby relieving the condition. The subject in need thereof can be, for example, a patient that had a liver disease and has experienced a traumatic bleeding. Also disclosed here in is a method of preventing a condition of neuron loss. In some embodiments, the method comprises administering ornithine in combination with at least one of phenylacetate and phenylbutyrate to a subject in need thereof, and thereby preventing the condition. The subject in need thereof can be, for example, a patient having a liver disease that is expected to experience a traumatic bleeding.

In some embodiments, the liver disease is a chronic liver disease. In some embodiments, the liver disease is hepatic encephalopathy. In some embodiments, the liver disease is cirrhosis. In some embodiments, the liver disease is minimal hepatic encephalopathy.

In some embodiments, the traumatic bleeding is caused by a surgical procedure for treating the liver disease. For example, the surgical procedure can be liver transplantation. In some embodiments, the traumatic bleeding is caused by traumatic injury.

In some embodiments, at least one symptom of the condition of neuron loss is decreased count of neurons in the subject. In some embodiments, at least one symptom of the condition of neuron loss is decreased count of neurons in the frontal cortex of the subject. In some embodiments, at least one symptom of the condition of neuron loss is decreased count of functional neurons in the subject. In some embodiments, at least one symptom of the condition of neuron loss is decreased count of functional neurons in the frontal cortex of the subject. In some embodiments, the condition of neuron loss is caused by hypotension.

In some embodiments, the treatment of the condition is achieved by reducing the level of one or more cellular stress proteins in the subject. In some embodiments, the prevention of the condition is achieved by reducing the level of one or more cellular stress proteins in the subject. In some embodiments, at least one of the one or more cellular stress proteins is hsp32, hsp70 or caspase-3. In some embodiments, the treatment of the condition is achieved by reducing apoptotic cell death in the subject. In some embodiments, the prevention of the condition is achieved by reducing apoptotic cell death in the subject.

Some embodiments disclosed herein provide a method of treating a condition of neuron loss. The method, in some embodiments, comprises administering ornithine in combination with at least one of phenylacetate and phenylbutyrate to a subject in need thereof, and thereby relieving the condition. In some embodiments, the subject in need thereof is a patient having a liver disease that has been treated by liver transplantation. In some embodiments, the subject is suffering from or at a risk of developing hypotension. Also provided herein is a method of preventing a condition of neuron loss. The method, in some embodiments, comprises administering ornithine in combination with at least one of phenylacetate and phenylbutyrate to a subject in need thereof, and thereby preventing the condition. In some embodiments, the subject in need thereof is a patient having a liver disease that is going to be treated by liver transplantation. In some embodiments, the subject is suffering from or at a risk of developing hypotension.

In some embodiments, the liver disease is a chronic liver disease. In some embodiments, the chronic liver disease is cirrhosis. In some embodiments, the hypotension is caused by blood loss. In some embodiments, the hypotension is caused by a traumatic bleeding. In some embodiments, the hypotension is caused by a surgical procedure, for example a surgical procedure for treating a liver disease. In some embodiments, the subject is suffering from a liver disease. In some embodiments, the subject is suffering from a chronic liver disease. In some embodiments, the subject is also suffering from minimal hepatic encephalopathy.

In some embodiments, at least one symptom of the condition of neuron loss is decreased count of neurons in the subject. In some embodiments, at least one symptom of the condition of neuron loss is decreased count of neurons in the frontal cortex of the subject. In some embodiments, at least one symptom of the condition of neuron loss is decreased count of functional neurons in the subject. In some embodiments, at least one symptom of the condition of neuron loss is decreased count of functional neurons in the frontal cortex of the subject.

In some embodiments, the condition of neuron loss is caused by hypotension. In some embodiments, the treatment or prevention of the condition is achieved by reducing the level of one or more cellular stress proteins in the subject. In some embodiments, at least one of the one or more cellular stress proteins is hsp32, hsp70 or caspase-3. In some embodiments, the treatment or prevention of the condition is achieved by reducing apoptotic cell death in the subject.

In the methods disclosed herein, in some embodiments, separate pharmaceutically acceptable salts of the ornithine and at least one of phenylacetate and phenylbutyrate are administered to the subject. In some embodiments, the at least one of phenylacetate and phenylbutyrate is administered as a sodium phenylacetate or sodium phenylbutyrate. In some embodiments, the ornithine is administered as a free monomeric amino acid or physiologically acceptable salt thereof. In some embodiments, the ornithine and phenylacetate is administered as ornithine phenylacetate. In some embodiments, the administration is oral, intravenous, intraperitoneal, intragastric, or intravascular administration. In some embodiments, the administration the administration is intravenous administration. In some embodiments, the administration the administration is oral administration.

### BRIEF DESCRIPTION OF THE DRAWINGS

FIG. 1 is a schematic illustration of the experiment design described in Example 4.

FIGS. 2A-C show neuronal count, NeuN levels (detected by immunofluorescence and western blot) in SHAM and BDL rats with or without induced hypotension.

FIG. 3 shows images of NeuN staining in SHAM and BDL rats with induced hypotension at blood pressure of 60 mmHg.

FIGS. 4A-B show level of cleaved caspase 3 in SHAM and BDL rats with no induced hypotension, or with induced hypotension at blood pressure of 60 mmHg.

FIG. 5 show ammonia levels in SHAM, BDL rats and BDL rats treated with OP.

FIG. 6 show behavioural testing results of SHAM, BDL rats and BDL rats treated with OP.

FIGS. 7A-C show neuronal count, NeuN level, cleaved caspase-3 level in SHAM rats, BDL rats and BDL rats treated with OP, where the BDL rats and the BDL rats treated with OP have been induced for hypotension at blood pressure of 60 mmHg.

### DETAILED DESCRIPTION

In the following detailed description, reference is made to the accompanying drawings, which from a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

### DEFINITIONS

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e. Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to the combined amount of the active ingredients (e.g., ornithine and phenylacetate or phenylbutyrate).

As used herein, a “unit dosage” refers to an amount of therapeutic agent administered to a patient in a single dose.

As used herein, a “daily dosage” refers the total amount of therapeutic agent administered to a patient in a day.

As used herein, “therapeutically effective amount” or “pharmaceutically effective amount” is meant an amount of therapeutic agent, which has a therapeutic effect. The dosages of a pharmaceutically active ingredient which are useful in treatment are therapeutically effective amounts. Thus, as used herein, a therapeutically effective amount means an amount of therapeutic agent which produces the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The term “phenylacetate” as used herein, refers to the anionic form of phenylacetic acid with the following chemical structure:

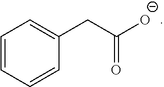

The term “L-ornithine phenylacetate” as used herein, refer to a compound consisting of L-ornithine cation and phenylacetate anion. It has the following chemical structure:

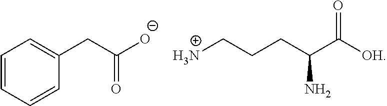 [SMILES: O=C([O-])Cc1ccccc1 ; N[C@@H](CCC[NH3+])C(=O)O]

The term “phenylbutyrate” as used herein, refers to the anionic form of phenylbutyric acid with the following chemical structure:

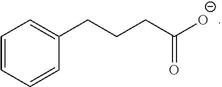 [SMILES: O=C([O-])CCCc1ccccc1]

The term “L-ornithine phenylbutyrate” as used herein, refers to a compound consisting, of L-ornithine cation and phenylbutyrate anion. It has the following chemical structure:

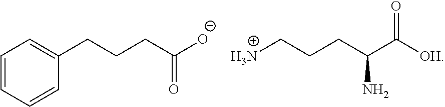 [SMILES: O=C([O-])CCCc1ccccc1 ; N[C@@H](CCC[NH3+])C(=O)O]

### ABBREVIATIONS

BDL=bile duct ligation;

OP=ornithine, phenylacetate;

HE=hepatic encephalopathy

MHE=minimal hepatic encephalopathy

LT=liver transplantation

### Neuronal Cell Loss

Neurons, also known as neuronal cells, are highly specialized cells of the nervous system. Neurons are electrically excitable cells that process and transmit information through electrical and chemical signals. Neurons are the core components of the brain and spinal cord of the central nervous system (CNS), and of the ganglia of the peripheral nervous system (PNAS). There are a variety of types of neurons, including and not limited to, sensory neurons, motor neurons, and interneurons (or associative neurons). Non-limiting symptoms of neuron loss (also known as neurodegeneration.) can be death of neurons, decrease in the count of neuron cells, decrease in functional neuron cells, loss of structure of neurons, loss of function of neurons, decrease in neuronal differentiation, decrease in neuronal proliferation, or any combination thereof. There are many causes for neuron loss. For example, neuron loss can be caused by aging, disease (for example and not limited to, neurodegenerative diseases and liver diseases), exposure to neurotoxic chemicals, injury, inactivity, or any combination thereof.

A condition of neuronal cell loss can have various symptoms, including but not limited to, decreased count of neurons in the subject (e.g., in the frontal cortex of the subject), and decreased count of functional neurons in the subject (e.g., in the frontal cortex of the subject).

Liver diseases are often accompanied with neuropsychiatric complications characterized by cognitive and motor dysfunction. The only curative treatment for end-stage liver disease and hepatic encephalopathy (HE) to date remains to be liver transplantation (LT). It was found that even following the implantation of a new liver, persisting neurological complications remain a common problem affecting many liver transplant recipients. Liver transplantation is a major surgical procedure accompanied by intraoperative stress and confounding factors, including blood loss and hypotension. Other types of traumatic bleeding in liver disease patients can also lead to blood loss and hypotension, which in some instances results in neuron loss in the patients. The traumatic bleeding can be caused by, for example, surgeries (for example, a surgical procedure for treating a liver disease (e.g., liver transplantation, partial liver transplantation, liver resection, and endoscopy)) and injuries. Non-limiting types of wounds that may be caused by the injuries include abrasion, excoriation, hematoma, laceration, incision, puncture wound, contusion, crushing injuries, and ballistic trauma. There is a need for therapies for preventing and/or relieving neurological complications (e.g., neuron loss) in the liver disease patients after receiving LT treatment or suffering other types of traumatic bleeding. Without being bound by any particular theory, it is believed that in a patient of minimal HE (MHE), the compromised brain may become susceptible to hypotensive insults, resulting in cell injury and death (e.g., injury and death to neuronal cells).

Traumatic bleeding oftentimes leads to blood loss. The extent of blood loss in person who is experiencing or has experienced a traumatic bleeding can vary. For example, the person cart lose, or lose about, 1%, 5%, 10%, 12%, 15%, 18%, 20%, 22%, 25%, 28%, 30%, 32%, 35%, 38%, 40%, 45%, 50%, 60%, 70%, 80%, or a range between any two of these values, of the total blood volume or circulating blood volume of that person because of the traumatic bleeding. In some embodiments, the traumatic bleeding results in class I hemorrhage which involves loss of up to 15% of total blood volume of the person. In some embodiments, the traumatic bleeding can result in class II hemorrhage which involves loss of about 15%-30% of total blood volume of the person. In some embodiments, the traumatic bleeding can result in class III hemorrhage which involves loss of about 30%-40% of circulating blood volume of the person. In some embodiments, the traumatic bleeding can result in class IV hemorrhage which involves loss of greater than 40% of circulating blood volume of the person. The person can, for example, lose, or lose about, 50 mL (milliliter), 60 mL, 90 mL, 100 mL, 150 mL, 200 mL, 300 mL, 400 mL, 500 mL, 600 mL, 700 mL, 800 mL, 900 mL, 1000 mL, 1100 mL, 1200 mL, 1300 mL, 1400 mL, 1500 mL, 1600 mL, 1700 mL, 1800 mL, 1900 mL, 2000 mL, 2100 mL, 2200 mL, 2300 mL, 2400 mL, 2400 mL, 2500 mL, 2800 mL, 3000 mL, 3500 mL, 4000 mL, or a range between any two of these values, of blood. In some embodiments, the traumatic bleeding results in a blood loss of about 750 mL to about 2000 mL. The traumatic bleeding can, in some embodiments, lead to hypotension because of the blood loss. For example, because of the traumatic bleeding, the person's systolic blood pressure can be at 90 mmHg, 80 mmHg, 70 mmHg, 60 mmHg, 50 mmHg, 40 mmHg, 30 mmHg, or a range between any two of these values. In some embodiments, the person's systolic blood pressure is below 90 mmHg, 80 mmHg, 70 mmHg, 60 mmHg, 50 mmHg, 40 mmHg, 30 mmHg, or 20 mmHg, or lower. In some embodiments, the person can have a diastolic blood pressure at 60 mmHg, 50 mmHg, 40 mmHg, 30 mmHg, 20 mmHg, 10 mmHg, 5 mmHg, or a range between any two of these values; or any combination thereof, because of the traumatic bleeding. In some embodiments, the person can have a diastolic blood pressure below 60 mmHg, 50 mmHg, 40 mmHg, 30 mmHg, 20 mmHg, 10 mmHg, or 5 mmHg, or lower.

Neuronal cell loss can be a symptom or a result of an underlying condition (e.g., a liver disorder, blood loss, or hypotension), and therefore a subject may have neuronal cell loss that is associated with a one or more conditions. In some embodiments, the neuronal cell loss is associated with a liver disease. Non-limiting examples of liver disease include intrahepatic cholestasis (alagille syndrome, biliary liver cirrhosis), fatty liver (alcoholic fatty liver, reye syndrome), hepatic vein thrombosis, hepatolentricular degeneration, hepatomegaly, liver abscess (amebic liver abscess), liver cirrhosis (e.g., alcoholic, biliary and experimental liver cirrhosis), alcoholic liver diseases (fatty liver, hepatitis, and cirrhosis), parasitic (hepatic echinococcosis, fascioliasis, amebic liver abscess), jaundice (hemolytic, hepatocellular, and cholestatic), cholestasis, portal hypertension, liver enlargement, ascites, hepatitis (alcoholic hepatitis, animal hepatitis, chronic hepatitis (e.g., autoimmune, hepatitis B, hepatitis C, hepatitis D, and drug induced), toxic hepatitis, viral human hepatitis (e.g., hepatitis A, hepatitis B, hepatitis C, hepatitis D, and hepatitis E), Wilson's disease, granulomatous hepatitis, secondary biliary cirrhosis, hepatic encephalopathy (e.g., minimal hepatic encephalopathy), varices, primary biliary cirrhosis, primary sclerosing cholangitis, hepatocellular adenoma, hemangiomas, bile stones, liver failure (hepatic encephalopathy, acute liver failure), and liver neoplasms (angiomyolipoma, calcified liver metastases, cystic liver metastases, epithelial tumors, fibrolamellar hepatocarcinoma, focal nodular hyperplasia, hepatic adenoma, hepatobiliary cystadenoma, hepatoblastoma, hepatocellular carcinoma, hepatoma, liver cancer, liver hemangioendothelioma, mesenchymal hamartoma, mesenchymal tumors of liver, nodular regenerative hyperplasia, benign liver tumors (hepatic cysts [simple cysts, polycystic liver disease, hepatobiliary cystadenoma, choledochal cyst], mesenchymal tumors [mesenchymal hamartoma, infantile hemangioendothelioma, hemangioma, peliosis hepatis, lipomas, inflammatory pseudotumor, miscellaneous], Epithelial tumors [bile duct epithelium (bile duct hamartoma, bile duct adenoma), hepatocyte (adenoma, focal nodular hyperplasia, nodular regenerative hyperplasia)], malignant liver tumors [hepatocellular, hepatoblastoma, hepatocellular carcinoma, cholangiocellular, cholangiocarcinoma, cystadenocarcinoma, tumors of blood vessels, angiosarcoma, Karposi's sarcoma, hemangioendothelioma, other tumors, embryonal sarcoma, fibrosarcoma, leiomyosarcoma, rhabdomyosarcoma, carcinosarcoma, teratoma, carcinoid, squamous carcinoma, primary lymphoma]), peliosis hepatis, erythrohepatic porphyria, hepatic porphyria (acute intermittent porphyria, porphyria cutanea tarda), Zellweger syndrome).

In some embodiments, the neuron loss is associated with a chronic liver disease, for example hepatitis or cirrhosis. Hypotension can be a major complication in cirrhosis patients that have gone through a surgical procedure (e.g., liver transplantation) or suffered a traumatic bleeding. Without being bound by any particular theory, it is believed that blood loss and hypotension that are associated with a surgical procedure or a traumatic bleeding can lead to injury and/or death of neuronal cells. A condition of neuron loss can be, but is not necessarily, associated with blood loss and/or hypotension. In some embodiments, the condition of neuron loss is caused by hypotension. In some embodiments, the condition of neuron loss is associated with hypotension. In some embodiments, the condition of neuron loss is caused by blood loss. In some embodiments, the condition of neuron loss is associated with blood loss. In some embodiments, the subject receiving treatment for neuron loss is a patient that has received a surgical procedure (e.g., liver transplantation) for treating a liver disease (e.g., an end-stage liver disease). In some embodiments, the subject receiving treatment for preventing neuron loss is a patient suffering a liver disease (e.g., an end-stage liver disease) and is going to receive a surgical procedure for treating the liver disease (e.g., liver transplantation). In some embodiments, the patient is suffering from or at the risk of developing hypotension (e.g., perioperative hypotension). In some embodiments, the patient is suffering from one or more neurological complications associated with a surgical procedure for treating a liver disease (e.g., liver transplantation). In some embodiments, the patient is at the risk of developing one or more neurological complications associated with a surgical procedure for treating a liver disease (e.g., liver transplantation).

Neuronal cells can be observed and quantified by using various neuron-specific markers known in the art, including but not limited to, Neuronal Nuclei (NeuN), Neuron specific enolase (NSF), βIII Tubulin (TuJ1), Doublecortin (DCX), and c-fos. Neuron loss can be determined by many methods known in the art. For example, various viability assays, mostly for in vitro applications, can be used to measure neuronal cell death. Non-limiting examples of the viability assays include lactate dehydrogenase (LDH) release assay which measures the amount of the cytoplasmic enzyme released into the bathing medium, trypan blue and propidum iodide assays which measure the ability of cells to exclude dye from their cytoplasm, 3-(4,5-dimethylthiazol-2-yl)-2,5-diphenyltetrazolium bromide (MTT) assay which measures the mitochondrial activity of viable cells by quantifying the conversion of the tetrazolium salt to its formazan product, and an assay details the measurement of luciferase expression as an indication of neuronal viability within a relatively small population of transfected neurons. See e.g., Aras et al. Current Protocols in Neuroscience (2008). Apoptotic analysis (e.g., western blot), immunohistochemistry assays, and enzyme assays showing a change in caspase-9-like and caspase-3-like activities in neuronal cells, can also be used to measure neuron loss. A number of other assays, for example an xCELLigence system based on impedance measurement (Diemert et al., J. Neurosci. Methods, 203(1):69-77 (2012)), can be used for real-time detection of neuronal cell death. In some embodiments, neuron loss is determined by an immunohistochemistry assay.

### Treatment and Prevention of Neuronal Cell Loss

Some embodiments disclosed herein include methods of treating or preventing a condition of neuron loss by co-administering to a subject in need thereof ornithine in combination with phenylacetate and/or phenylbutyrate. Some such embodiments include therapeutic treatment, and some embodiments include prophylactic treatment.

The subject in need thereof can be a patient who is suffering from a condition of neuron loss or a subject that is suspect of or at the risk of developing a condition of neuron loss. The subject may have, or may not have, symptoms of liver diseases (for example, acute liver failure or acute liver decompensation). In some embodiments, the subject is suffering from a liver disease, for example a chronic liver disease. In some embodiments, the subject does not have hepatic encephalopathy (HE). In some embodiments, the subject has a liver disease but is not exhibiting any significant symptoms of liver disease. In some embodiments, the subject is a patient of liver disease that has been treated by a surgical procedure for treating the liver disease (e.g., liver transplantation, partial liver transplantation, liver resection, and endoscopy). In some embodiments, the subject is a patient of HE that has been treated by a surgical procedure for treating HE (e.g., liver transplantation). In some embodiments, the subject is a patient of HE that is going to be treated by a surgical procedure for treating the liver disease (e.g., liver transplantation). In some embodiments, the subject is a patient of minimal HE (MHE) that has been treated by a surgical procedure for treating the liver disease (e.g., liver transplantation). In some embodiments, the subject is a patient of MHE that is going to be treated by a surgical procedure for treating the liver disease (e.g., liver transplantation). The subject in need thereof can also be a patient of chronic liver disease that has been treated by a surgical procedure for treating the liver disease (e.g., liver transplantation), or a patient of chronic liver disease that is going to be treated by a surgical procedure for treating the liver disease (e.g., liver transplantation). The subject in need thereof may, or may not, suffer from hypotension. In some embodiments, the subject in need thereof suffers from blood loss, for example blood loss caused by a traumatic bleed (e.g., a surgical procedure). In some embodiments, the subject is suffering from MHE and hypotension. In some embodiments, the subject is suffering from HE and hypotension. In some embodiments, the subject is suffering from a liver disease (e.g., a chronic liver disease) and hypotension. The hypotension can be, for example, less than 90 millimeters of mercury (mmHg), 80 mmHg, 70 mmHg, 60 mmHg, 50 mmHg, 40 mmHg, or 30 mmHg for systolic blood pressure. The hypotension can also be, for example, less than 60 mmHg, 50 mmHg, 40 mmHg, 30 mmHg, 20 mmHg, 10 mmHg for diastolic blood pressure. In some embodiments, the subject in need thereof has a systolic blood pressure at 90 mmHg, 80 mmHg, 70 mmHg, 60 mmHg, 50 mmHg, 40 mmHg, 30 mmHg, or a range between any two of these values; has a diastolic blood pressure at 60 mmHg, 50 mmHg, 40 mmHg, 30 mmHg, 20 mmHg, 10 mmHg, or a range between any two of these values; or any combination thereof. In some embodiments, the subject in need thereof has a systolic blood pressure at or below 90 mmHg, 80 mmHg, 70 mmHg, 60 mmHg, 50 mmHg, 40 mmHg, 30 mmHg, or 20 mmHg; has a diastolic blood pressure at or below 60 mmHg, 50 mmHg, 40 mmHg, 30 mmHg, 20 mmHg, 10 mmHg, 5 mmHg, or a range between any two of these values; or any combination thereof.

The methods disclosed herein can comprise identifying a subject in need thereof as described herein. In some embodiments, the subject is suffering from hypotension. The hypotension can be caused by, for example, blood loss or traumatic bleeding. In some embodiments, the hypotension is caused by a surgical procedure, for example a surgical procedure for treating a liver disease. In some embodiments, the subject is a patient that had a liver disease and suffered a traumatic bleeding. In some embodiments, the subject is a patient having a liver disease and is at risk of or expected to experience traumatic bleeding. The traumatic bleeding can be caused, for example, by a surgical procedure (e.g., liver transplantation) or a traumatic injury. The traumatic bleeding, in some embodiments, can lead to blood loss and/or hypotension.

In some embodiments, the method comprises identifying a subject suffering from a condition of neuronal cell loss or a subject that is suspect of or at the risk of developing a condition of neuronal cell loss; and co-administering to the subject ornithine in combination with phenylacetate and/or phenylbutyrate. In some embodiments, the methods disclosed herein include acquiring knowledge of the presence of a condition of neuronal cell loss in a subject or the risk/potential of developing a condition of neuronal cell loss in a subject; and co-administering to the subject ornithine in combination with phenylacetate and/or phenylbutyrate. In some embodiments, the method comprises identifying a subject suffering from hypotension.

Change in neuronal cell loss, for example attenuation or acceleration of neuronal cell loss, can be detected, for example, by detecting change in the number of neuronal cell death, detecting change in the structure of neuronal cells, detecting change in the functions of neuronal cells, or any combination thereof of the subject. The neuronal cell loss can be, for example, reduction in the total number of neuronal cells, reduction in the number of functional neuronal cells, reduction in the number of live neuronal cells, or any combination thereof. The neuronal cell loss can be caused by, for example, increase in the death of neuronal cells, increase in the death of functional neuronal cells, decrease in differentiation or proliferation of neuronal cells, decrease in differentiation or proliferation of functional neuronal cells, decrease in differentiation or proliferation of precursor cells of neuronal cells, or any combination thereof.

Some embodiments disclosed herein provide methods of treating or preventing a condition of neuronal cell loss by co-administering to a subject in need thereof ornithine in combination with phenylacetate and/or phenylbutyrate. Some embodiments can include identifying a subject as having or at risk for developing a condition of neuronal cell loss prior to administering the ornithine in combination with phenylacetate and/or phenylbutyrate.

By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

In some embodiments, the co-administration is useful to reduce the level of one or more cellular stress proteins in the subject, which treat or reduce the likelihood of the death of neuronal cells (e.g., apoptosis of neuronal cells). Non-limiting examples of cellular stress proteins include heat shock proteins (hsp) is (e.g., hsp27, hsp32, hsp40, hsp60, hsp70, hsp90, and hsp105); and caspases (e.g., caspase-3, caspase-7 and caspase-9). In some embodiments, at least one of the one or more cellular stress proteins is hsp32, hsp70 and capsase-3. In some embodiments, neuronal cell loss is attenuated or prevented in patients with an existing chronic liver disease such as cirrhosis by the administration of the combination. Thus, in some embodiments, the combination is administered to a patient having a chrome liver disease also having a condition of neuronal cells loss. In some embodiments, the combination is administered to a patient having a chronic liver disease also having a condition of hypotension. In some embodiments, the treatment and/or prevention of the condition of neuronal cell death is achieved by reducing apoptotic cell death in the subject.

While not being bound by any particular theory, in some embodiments, the co-administration prevents or relieves the condition of neuronal cell loss through effects on preventing or reducing apoptosis. In some embodiments, reducing apoptosis results in the treating or prevention of the condition of neuronal cell loss.

In some embodiments, the methods and compositions disclosed herein can prevent and/or reduce loss of neuronal cells (e.g., functional neuronal cells). The neuronal cells can be, for example, neuronal cells in one or two specific regions of the subject, including but not limited to, frontal cortex, visual cortex, cerebellar cortex, cerebral cortex, motor cortex, oculomotor nuclei, cerebellum, and basal ganglia. The methods and compositions can, for example, prevent or delay the onset of the loss of neuronal cells. In some embodiments, the loss of neuronal cells s prevented from occurring. In some embodiments, the onset of loss of neuronal cells is delayed. The delay can be, for example, days, weeks or months. In some embodiments, the onset of loss of neuronal cells is delayed by at least, or at least about, one, two, three, four, five, six, seven, eight, nine, ten, or more weeks. In some embodiments, the onset of loss of neuronal cells is delayed by at least, or at least about, one, two, three, four, five, six, seven, eight, nine, ten, or more months.

The methods and compositions disclosed herein can, in some embodiments, reduce the rate of neuron loss. The neuron loss can be, for example, the loss in the total number of neurons, the loss in the number of functional neurons, or any combination thereof. In some embodiments, the rate of loss of neuronal cells in a patient receiving or having received treatment by the methods and/or compositions disclosed herein is reduced by at least, or at least about, 5%, 10%, 20%, 30%, 40%, 50%, 60%, 70%, 80%, 90%, 95%, 98%, or 99% as compared to patients receiving no treatment. In some embodiments, the methods and/or compositions reduce the rate of loss of neuronal cells in the patient receiving or having received treatment by, or by about, 5%, 10%, 20%, 30%, 40%, 50%, 60%, 70%, 80%, 90%, 95%, 98%, or 99%, or a range between any two of these values as compared to patients receiving no treatment. In some embodiments, the methods and composition reduce the final loss in neuronal cells. For example, the final loss of neuronal cells in the patient receiving or having received treatment can be at most, or at most about, 1%, 3%, 5%, 10%, 20%, 30%, 40%, 50%, 60%, 70%, 80%, 90%, 95%, 98%, or 99% of the final loss of neuronal cells in patients received no treatment. In some embodiments, the final loss of neuronal cells in the patient receiving or having received treatment is, or is about, 1%, 1%, 5%, 10%, 20%, 30%, 40%, 50%, 60%, 70%, 80%, 90%, 95%, 98%, or 99%, or a range between any two of these values, of the final loss of neuronal cells in patients receiving no treatment.

In some embodiments, the methods and composition reduce the final loss in neuronal cells so that the total number of neuronal cells or the number of functional neuronal cells in the patient receiving or having received treatment is at least, or at least about, 5%, 10%, 20%, 30%, 40%, 50%, 60%, 70%, 80%, 90%, 100%, 150%, 200%, or more, higher than the total number of neuronal cells or the number of functional neuronal cells in patients receiving no treatment. In some embodiments, the methods and composition may reduce the final loss in neuronal cells so that the total number of neuronal cells or the number of functional neuronal cells in the patient receiving or having received treatment is 5%, 10%, 20%, 30%, 40%, 50%, 60%, 70%, 80%, 90%, 100%, 150%, 200%, 300%, 400%, 500%, 600%, 700%, 800%, 900%, or a range between any two of these values, higher than the total number of neuronal cells or the number of functional neuronal cells in patients received no treatment.

### Salts

In some embodiments, the ornithine and phenylacetate or phenylbutyrate are administered as pharmaceutically acceptable salts. The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable salts can also be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297 published Sep. 11, 1987 (incorporated by reference herein in its entirety).

In some embodiments, ornithine is administered as the ornithine HCl salt. In some embodiments, phenylacetate or phenylbutyrate is administered as their sodium salts. In some embodiments, ornithine and phenylacetate or phenylbutyrate are administered as salts of each other (e.g., ornithine phenylacetate).

### Pharmaceutical Compositions and Methods of Administration

The ornithine (e.g., L-ornithine) and phenylacetate or phenylbutyrate may be administered separately or in a single dosage form. In one embodiment, the combination is administered as the ornithine phenylacetate salt or as a solution of the ornithine phenylacetate salt.

Different forms of composition of ornithine in combination with at least one of phenylacetate (or phenyl acetate salts) and phenylbutyrate have been described in U.S. Patent Publication Nos. US2008/0119554 and US2010/0280119, which are hereby incorporated by reference in their entireties. In some embodiments, ornithine and phenylacetate is present and/or administered as ornithine phenyl acetate or physiologically acceptable salt thereof. In some embodiments, ornithine is present and/or administered as a free monomeric amino acid or physiologically acceptable salt thereof In some embodiments, at least one of phenylacetate and phenylbutyrate is present and/or administered as a sodium phenylacetate or sodium phenylbutyrate. In some embodiments, a physiologically acceptable salt of ornithine and a physiologically acceptable salt of at least one of phenylacetate and phenylbutyrate are administered to the subject.

As disclosed herein, the ornithine and the phenylacetate and/or phenylbutyrate can be formulated for administration in a pharmaceutical composition comprising a physiologically acceptable surface active agents, carriers, diluents, excipients, smoothing agents, suspension agents, film forming substances, coating assistants, or a combination thereof. In some embodiments, the ornithine and the phenylacetate and/or phenylbutyrate are formulated for administration with a pharmaceutically acceptable carrier or diluent. The ornithine and the phenylacetate and/or phenylbutyrate can be formulated as a medicament with a standard pharmaceutically acceptable carrier(s) and/or excipient(s) as is routine in the pharmaceutical art. The exact nature of the formulation will depend upon several factors including the desired route of administration. Typically, ornithine and the phenylacetate and/or phenybutyrate are formulated for oral, intravenous, intragastric, intravascular or intraperitoneal administration. Standard pharmaceutical formulation techniques may be used, such as those disclosed in Remington's The Science and Practice of Pharmacy, 21st Ed., Lippincott Williams & Wilkins (2005), incorporated herein by reference in its entirety.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid, emulsifiers, such as the TWEENS, wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 0.1 mg/kg and 4000 mg/kg body weight, preferably between about 80 mg/kg and 1600 mg/kg body weight. Alternatively dosages may be based and calculated upon the surface area of the patient, as understood by those of skill in the art.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on many factors including the subject being treated, the severity of the affliction, the manner of administration, the judgment of the prescribing physician. The compound or combination of compounds disclosed herein may be administered orally or via injection at a dose from 0.1 mg/kg to 4000 mg/kg of the patient's body weight per day. The dose range for adult humans is generally from 1 g to 100 g/day. Tablets or other forms of presentation provided in discrete units may conveniently contain an amount of the compound or combination of compounds disclosed herein which is effective at such dosage or as a multiple of the same, for instance, units containing 1 g to 60 g (for example, from about 5 g to 20 g, from about 10 g to 50 g, from about 20 g to 40 g, or from about 25 g to 35 g). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity. A typical dose of ornithine, or of phenylacetate or phenylbutyrate can be from 0.02 g to 1.25 g per kg of body weight, for example from 0.1 g to 0.5 g per kg of body weight, depending on such parameters. In some embodiments, a dosage of ornithine, or of phenylacetate or phenylbutyrate can be from 1 g to 100 g, for example, from 10 g to 80 g, from 15 g to 60 g, from 20 g to 40 g, or from 25 g to 35 g. In some embodiments, the ornithine and phenylacetate/phenylbutyrate can be administered in a weight ratio from 10:1 to 1:10, for example, from 5:1 to 1:5, from 4:1 to 1:4, from 3:1 to 1:3, from 2:1 to 1:2, or about 1:1. A physician will be able to determine the required dosage of ornithine and of phenylacetate or phenylbutyrate for any particular subject.

The exact formulation, route of administration and dosage for the pharmaceutical compositions of the compound or combination of compounds disclosed herein can be chosen by the individual physician in view of the patient's condition. (See, e.g., Fingl et al. 1975, in “The Pharmacological Basis of Therapeutics,” which is hereby incorporated herein by reference, with particular reference to Ch. 1). Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for compounds have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In cases of administration of a pharmaceutically acceptable salt, dosages may be calculated as the free base. In some embodiments, the composition is administered 1 to 4 times per day. Alternatively the compositions of the compound or combination of compounds disclosed herein may be administered by continuous intravenous infusion, preferably at a dose of each active ingredient up to 100 g per day. As will be understood by those of skill in the art, in certain situations it may be necessary to administer the compound disclosed herein in amounts that exceed, or even far exceed, the above-stated, preferred dosage range in order to effectively and aggressively treat particularly aggressive diseases or infections. In some embodiments, the compound or combination of compounds disclosed herein will be administered for a period of continuous therapy, for example for a week or more, or for months or years.

In some embodiments, the dosing regimen of the compound(s) or combination of compounds disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of the compound(s) or combination of compounds disclosed herein can be administered three times a day, twice a day, daily, every other day, three times a week, every other week, three times per month, once monthly, substantially continuously or continuously.

### EXAMPLES

Embodiments of the present application are disclosed in further detail in the following examples, which are not in any way intended to limit the scope of the present disclosure.

### Example 1

### In Vivo Effect in BDL Rats

Six-week bile-duct ligated (BDL) rats with MHE and respective controls (SHAM) were used. Blood was withdrawn from the femoral artery (inducing hypovolemia) until a mean arterial pressure of 30 and 60 mmHg (hypotension) and maintained for 120 minutes. Cerebral blood flow (BCF) was assessed by injecting fluorescent microspheres (1×10⁶ microspheres/ml) through the brachial artery. Upon sacrifice, brains were extracted for apoptotic analysis (western blot) and neuronal cell count (immunohistochemistry). In a separate group, BDL rats were treated for MHE with ornithine phenylacetate (OP; OCR-002) (1 g/kg) for 3 weeks.

Both BDL rats and SHAM-operated controls without hypotension did not display any cell injury or neuronal loss. However, BDL rats following hypotension (30 and 60 mmHg) demonstrated a significant decrease in neuronal cell count in the frontal cortex (using NeuN+DAPI and Cresyl Violet) compared to hypotensive SHAM-operated controls. In addition, neuronal loss was associated with an increased in cellular stress protein, hsp32, hsp70 and caspase-3, suggesting apoptotic cell death. CBF decreased in BDL rats compared to SHAM and correlated with degree of hypotension insult. BDL rats treated with OP did not lead to neuronal cell death following hypotension.

These results demonstrate that cirrhotic patients with MHE are more susceptible to hypotension-induced neuronal cell loss. Moreover, these results support that a patient with HE (for example MHE), with a “frail brain”, will fare worse during liver transplantation and consequently result in poor neurological outcome. Combination of MHE and hypotension may account for the persisting neurological complications observed in a number of cirrhotic patients following liver transplantation.

These results demonstrate a protective effect on neurons by OP in MHE patients, for example, to reduce the risk of neurological complications occurring post-LT. In addition, these data supports the use of ornithine phenylacetate in the treatment (including prevention) of neuron loss.

### Example 2

### Treatment of Neuron Loss in Hepatic Encephalopathy Patients That Have Been Treated With Liver Transplantation

This example is to determine whether treatment with L-ornithine phenylacetate combinations (OP) decreases neuron loss in hepatic encephalopathy (HE) patients that have been treated liver transplantation.

HE patients that have received liver transplantation are randomized to be administered, for example orally, with placebo or OP. Neuronal cell count for each of the patients is measured prior to and after being administered with placebo or OP. It is expected that the administration of OP is effective in reducing neuron loss in the patients.

### Example 3

### Treatment of Neuron Loss in Hepatic Encephalopathy Patients That Are Going to be Treated With Liver Transplantation

This example is to determine whether treatment with L-ornithine phenylacetate combinations (OP) can prevent neuron loss in hepatic encephalopathy (HE) patients that are going to be treated liver transplantation.

HE patients that are going to receive liver transplantation are randomized to start receiving, for example oral, administration of placebo or OP, before the patients are treated with liver transplantation. Neuronal cell count for each of the patients is measured prior to and after being administered with placebo or OP, and prior to and after being treated with liver transplantation. It is expected that the administration of OP is effective in preventing neuron loss caused by liver transplantation in the patients.

### Example 4

### Prevention of Neuron Loss by OP Treatment

### Experimental Design and Analyses

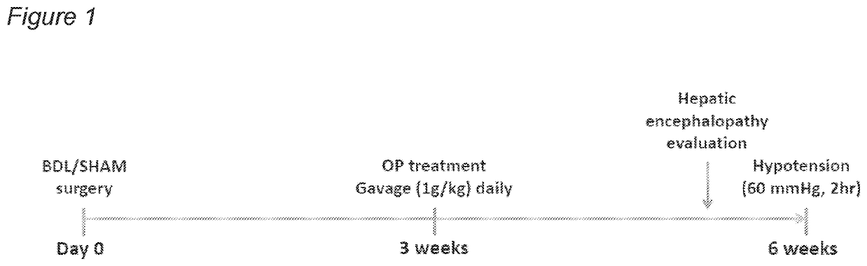

In this example, two hour hypotension was induced in 6-week BDL and SHAM operated rats after anesthetise. The 6-week BDL rats exhibited hyperammonemia, brain edema and impaired motor activity, spatial memory and learning deficits. In another set of animals, 3-week BDL rats were treated with ornithine phenylacetate (OP; 1 g/kg) by gavage for 3 weeks. Two-hour hypotension was then induced in OP-treated BDL rats and respective controls. All experiments were conducted following the Guidelines of Canadian Council on Animal Care. A schematic illustration of the experimental design is shown in FIG. 1.

Induction of hypotension: hypotension was induced by withdrawing blood from the femoral artery of the animals. First, Sprague-Dawley rats (175-200 g) were anesthetised with isoflurane to perform bile-duct ligation (BDL) or control-operations (SHAM) as previously described in Bosoi et al., Hepatology (2011) 53:1995-2002. The femoral artery was surgically exposed to insert a 24G catheter, which was then connected to a sphygmomanometer. This allowed blood to be removed from the animals while monitoring and maintaining the blood pressure (BP) of the animals at 30, 60 or 90 mmHg for 120 minutes. Body temperature of the tested animals was maintained at 37° C. with the use of a heat pad.

Tissue preparation: the rat brains were collected and frontal cortex of the animals was dissected and homogenized in lysis buffer (50 mM Tris, pH 7.5, 1 mM EDTA, 1/500 cold Protease Inhibitor Cocktail; Roche). Homogenates were centrifuged at 30,000 g for 40 minutes at 4° C. The supernatant was used as the brain cytosolic fraction. Protein content was determined according to the method described in Lowry et al., J Biol Chem (1951)193:265-275.

NeuN and cleaved caspase-3 expression: protein (20 ug) was loaded on 9% sodium dodecyl sulphate-polyacrylamide gel electrophoresis to be transferred on polyvinylidene difluoride membranes. After being blocked in 5% milk in TBS-T buffer (1 mM Tris pH 7.5, 10 mM NaCl and 0.5% Tween-20) for 1 hour at room temperature, the membrane was incubated in a dilution of 1:1000 of NeuN (EMD Millipore, Germany) or cleaved caspase 3 (Cell Signaling Technology, Danvers, Mass.) antibody in 5% milk-TBS-T buffer for 1 hour. NeuN is known to bind to neuron nuclei and cleaved caspase 3 is a well-known apoptosis marker. Membranes were washed 6 times in TBS-T buffer for 5 minutes and incubated 1 hour at room temperature with their corresponding secondary antibody coupled to horseradish peroxidase (1:10000). After 6 washes of 5 minutes in TBS-T, membrane were exposed to chemiluminescence reagent and probed on X-ray film. For control of protein loading, GAPDH (Sigma) was used at a dilution of 1:100000.

Immunofluorescence and histology: rats were perfused with saline and formalin 10% before brain extraction. Brain slices (50 μm) were made with a vibratome and were transferred in 24 well-plates containing PBS. Evaluation of neuronal cells in prefrontal cortex was made by immunofluorescence using NeuN antibody. First, brain slices were blocked with PBS-0.5% Triton X-100-10% donkey serum and were incubated for 30 minutes. After incubation, they were washed 3 times for 5 minutes in PBS. The slices were exposed to the first antibody (NeuN 1:200 or caspase-3 1:200 in blocking buffer) overnight at 4° C. After 3 washes, slices were then exposed to second antibody (mouse IgG coupled to Alexa488 fluorophore 1:200 or rabbit IgG coupled to Alexa594) in PBS-0.5% Triton X-100 and incubated for 30 minutes, in the dark, at room temperature. Following washes, (4′,6-diamidino-2-phenylindole) DAPI was added (1 μg/ml) and rinsed with PBS. Slices were then put on a microscope slide and mounting medium was added for fluorescence microscopy analysis (Zeiss). Staining of neurons was also obtained by cresyl violet staining (0.01%) followed by dehydration gradient in ethanol as described by Lange et al. Experimental Neurology (1999)158:254-260.

A number of behavioural tests were performed. For elevated-plus maze testing, 4 days before sacrifice, rats were placed in the elevated plus maze apparatus (box in which the maze is insert in: 100×100 cm, arms: 10×45 cm) for 10 minutes and evaluated for anxiety and explorative behaviours. Rats are conflicted by their explorative nature and their fear of open spaces. Closed arms provided the animal a safe environment compared to the open arms that provided values of exploration. Panlab tracking system was use quantify the time spent in open vs closed arms. In Open Field testing, on the day of sacrifice, rats were placed in an open field (arena: 90×90×40 cm with black plastic walls) in order to test for anxiety. As in the elevated-plus maze, rodents have innate fear for open areas and are also curious to explore a new environment. By allowing the rat to explore the arena for 10 minutes, this task allows evaluation by a tracking system (Panlab) the distance traveled (cm) and time spent (second, “s”) in wall areas compared to the middle or center area.

Ammonia level: plasmatic ammonia levels were assessed for (1) SHAM animals, (2) BDL animals, and (3) BDL animals treated with OP groups, and were measured using routine biochemistry techniques.

Statistical analysis: data were expressed as mean±standard error of the mean (SEM). Significance of difference was tested by ANOVA followed by Newman-Keuls or Tukey post-test using GraphPad Prism4. Probability values of p<0.05 were considered statistically significant.

### Results

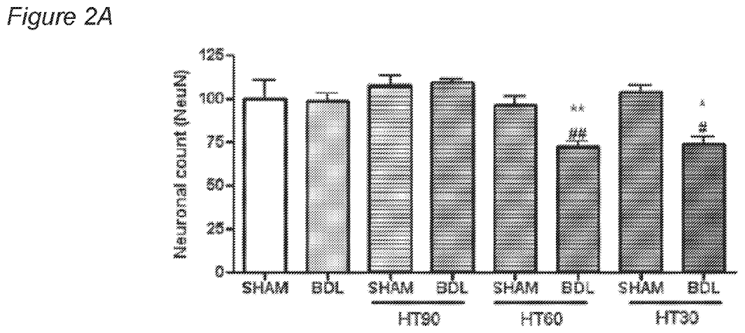

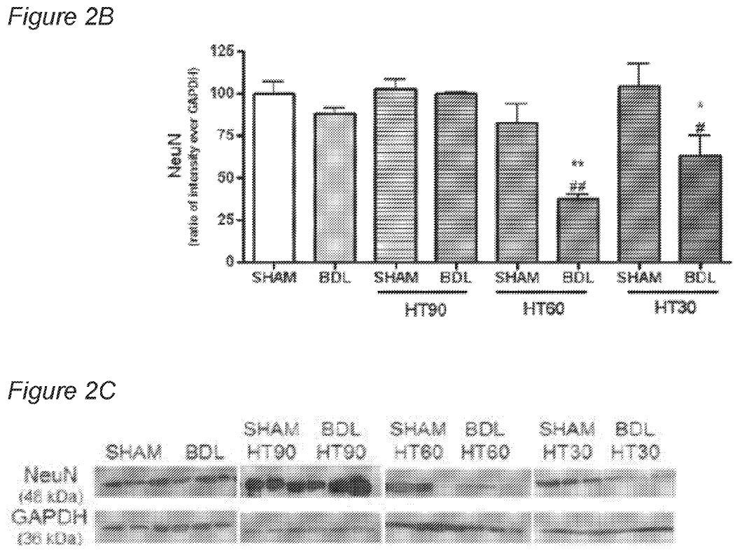

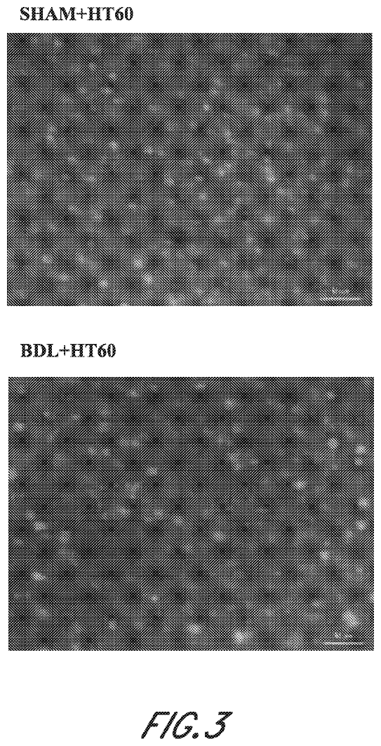

NeuN, a neuronal nuclear antigen, was used as a biomarker for measuring neuronal count. FIG. 2A shows the results of neuronal count based on NeuN staining, FIG. 2B shows ratio of intensity between NeuN staining and GAPDH staining (control), and FIG. 2C shows expression levels of NeuN and GAPDH proteins in rats in the absence of hypotension induction and in rats with induced hypotension at blood pressure of 30, 60 or 90 mmHg. FIG. 3 shows Immunofluorescence staining of NeuN protein in SHAM and BDL rats with an induced hypotension at blood pressure of 60 mmHg. As shown in FIGS. 2A-C and 3, SHAM and BDL rats had similar number of neurons without induced hypotension. Significant neuronal loss was observed in BDL rats following hypotension, for example BDL rats with an induced hypotension at blood pressure of 30 or 60 mmHg. In contrast, no significant neuronal loss was observed in SHAM operated rats with the induced hypotension (for example, at blood pressure of 30, 60 or 90 mmHg).

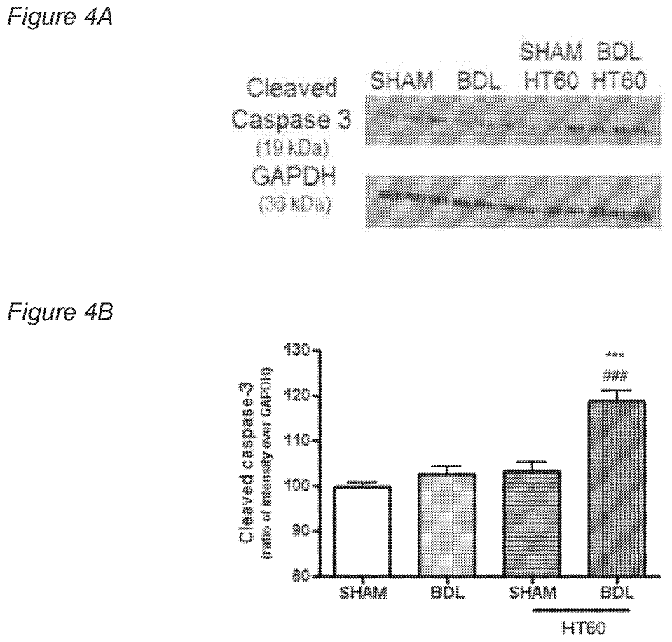

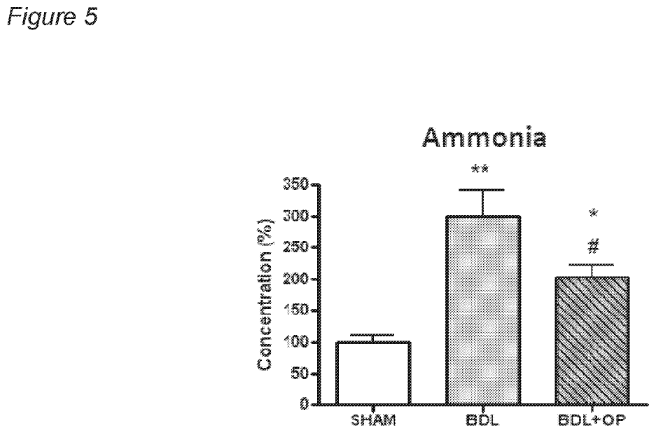

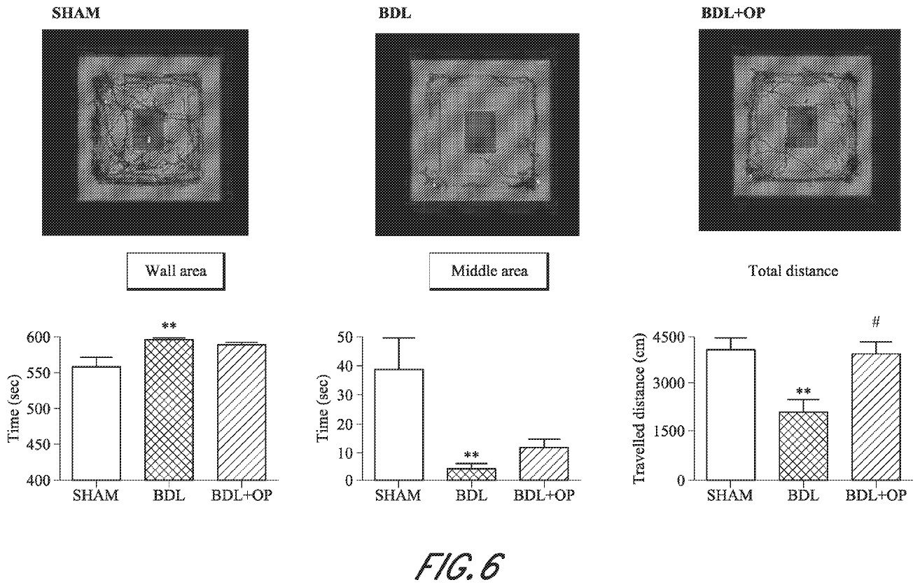

Caspase 3 has been found to be the predominant caspase involved in the cleavage of amyloid-beta 4A precursor protein, which is associated with neuronal death. The protein level of cleaved caspase 3 was measured to determine the neuronal loss in BDL and SHAM rats. As shown in FIGS. 4A-B, SHAM and BDL has comparable level of cleaved caspase-3; however, the level of cleaved caspase-3 increased significantly in BDL rats following induced hypotension at blood pressure of 60 mmHg.

Plasmatic ammonia levels were measured for (1) SHAM animals, (2) BDL animals, and (3) BDL animals treated with OP. The ammonia levels were significantly higher in BDL animals as compared to SHAM animals, and were reduced significantly in BDL animals after the OP treatment. FIG. 6 shows the results of behavioural tests performed in (1) SRAM, (2) BDL and (3) BDL animals treated with OP. FIG. 6 demonstrates beneficial effect of OP in treating MHE.

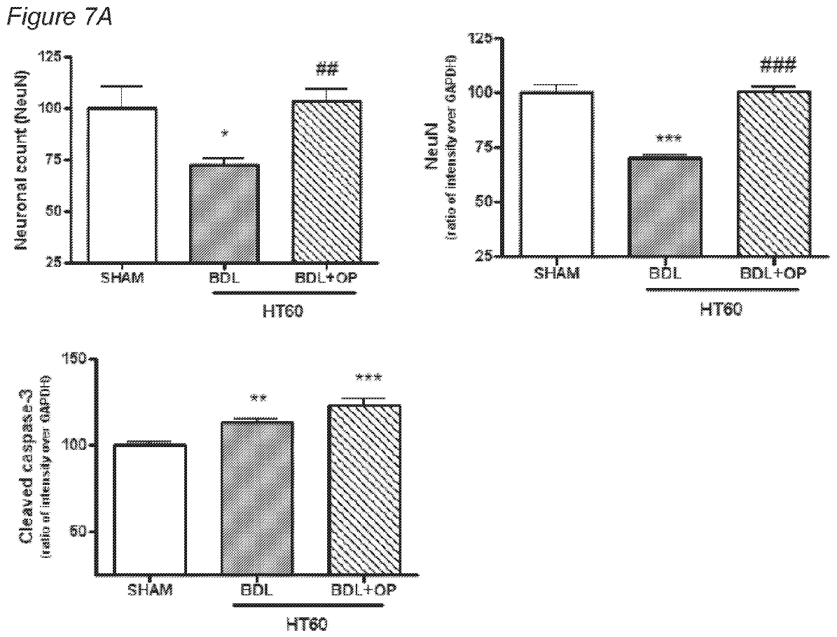

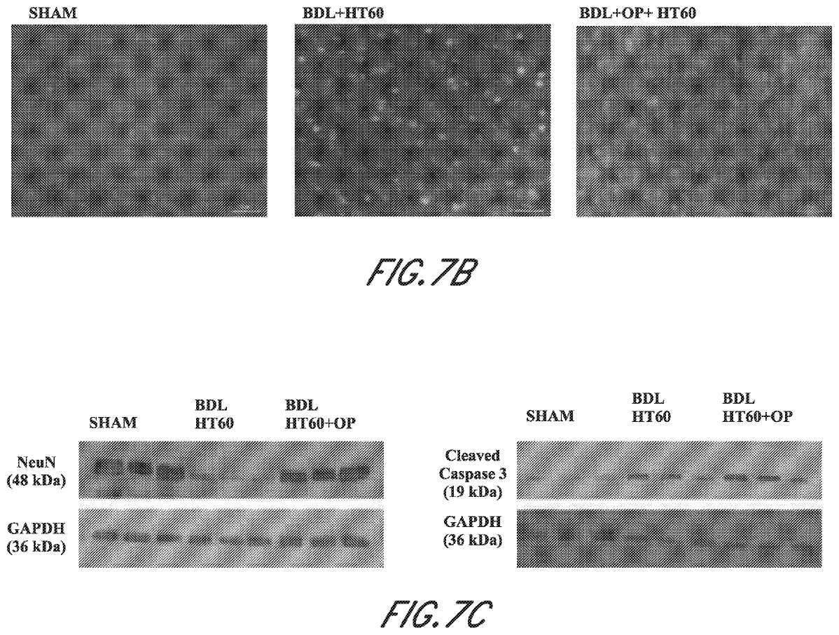

Neuronal count and neuronal loss were also determined by detecting NeuN and cleaved caspase 3, respectively, in (1) SHAM rats, (2) BDL rats with induced hypotension at blood pressure of 60 mmHg, and (3) BDL rats with induced hypotension at blood pressure of 60 mmHg and treated with OP. The results are shown in FIG. 7A. FIG. 7B shows immunofluorescence staining of NeuN protein in the same three groups of animals, and FIG. 7C shows western blot results for NeuN and cleaved caspase 3 protein levels in in the same three groups of animals. FIGS. 7A-C demonstrate that OP treatment prevents hypotension-induced neuronal loss.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent, to those skilled in the art. The various aspects and embodiments disclosed herein are for purposes of illustration and are not intended to be limiting, with the true scope and spirit being indicated by the following claims.

All references cited herein, including patents, patent applications, papers, text books, and the like, and the references cited herein, to the extent that they are not already, are hereby incorporated by reference in their entirety. In the event that one or more of the incorporated literature and similar materials differ from or contradict this application, including but not limited to defined terms, term usage, described techniques, or the like, this application controls.

1. A method of treating a condition of neuron loss, comprising administering ornithine in combination with at least one of phenylacetate and phenylbutvrate to a subject in need thereof, and thereby relieving the condition, wherein the subject is a patient that had a liver disease and has experienced a traumatic bleeding.

2. A method of preventing a condition of neuron loss, comprising administering ornithine in combination with at least one of phenylacetate and phenylbutvrate to a subject in need thereof, and thereby preventing the condition, wherein the subject is a patient having a liver disease that is expected to experience a traumatic bleeding, a patient having a liver disease that is going to be treated by liver transplantation, or a patient suffering from hypotension.

3. The method of claim 1, wherein the liver disease is a chronic liver disease.

4. The method of claim 1, wherein the liver disease is hepatic encephalopathy, cirrhosis or minimal hepatic encephalopathy.

5. (canceled)

6. (canceled)

7. The method of claim 1, wherein the traumatic bleeding is caused by a surgical procedure for treating the liver disease or by traumatic injury.

8. (canceled)

9. (canceled)

10. The method of claim 1, at least one symptom of the condition of neuron loss is one or more of decreased count of neurons in the subject, decreased count of neurons in the frontal cortex of the subject, decreased count of functional neurons in the subject, and decreased count of functional neurons in the frontal cortex of the subject.

11. (canceled)

12. (canceled)

13. (canceled)

14. The method of claim 1, wherein the condition of neuron loss is caused by hypotension.

15. The method of claim 1, wherein the treatment of the condition is achieved by reducing the level of one or more cellular stress proteins in the subject, or reducing apoptotic cell death in the subject.

16. The method of claim 2, wherein the prevention of the condition is achieved by reducing the level of one or more cellular stress proteins in the subject.

17. (canceled)

18. (canceled)

19. (canceled)

20. A method of treating a condition of neuron loss, comprising administering ornithine in combination with at least one of phenylacetate and phenylbutyrate to a subject in need thereof, and thereby relieving the condition, wherein the subject is a patient having a liver disease that has been treated by liver transplantation or a patient suffering from hypotension.

21. (canceled)

22. The method of claim 20, wherein the liver disease is a chronic liver disease.

23. (canceled)

24. (canceled)

25. (canceled)

26. The method of claim 20, wherein the hypotension is caused by blood loss.

27. The method of claim 2, wherein the subject is suffering from a liver disease.

28. (canceled)

29. (canceled)

30. The method of claim 20, at least one symptom of the condition of neuron loss is one or more of decreased count of neurons in the subject, decreased count of neurons in the frontal cortex of the subject, decreased count of functional neurons in the subject, and decreased count of functional neurons in the frontal cortex of the subject.

31. (canceled)

32. (canceled)

33. (canceled)

34. (canceled)

35. The method of claim 20, wherein the treatment of the condition is achieved by reducing the level of one or more cellular stress proteins in the subject, or by reducing apoptotic cell death in the subject.

36. (canceled)

37. (canceled)

38. The method of claim 1, wherein separate pharmaceutically acceptable salts of the ornithine and at least one of phenylacetate and phenylbutvrate are administered to the subject.

39. The method of claim 1, wherein the at least one of phenylacetate and phenylbutyrate is administered as a sodium phenylacetate or sodium phenylbutyrate.

40. The method of claim 1, wherein the ornithine is administered as a free monomelic amino acid or physiologically acceptable salt thereof.

41. The method of claim 1, wherein the ornithine and phenylacetate is administered as ornithine phenylacetate.

42. The method of claim 1, wherein the administration is oral, intravenous, intraperitoneal, intragastric, or intravascular administration.

43. (canceled)

44. (canceled)
